SEQUENCE LISTING (36 sequences)

SEQ ID NO: 1 - DNA, 297 bp, Escherichia coli
atggaaaaacgcacaccacatacacgtttgagtcaggttaaaaaacttgtcaatgccgggcaagttcgtacaacacgtagtgccctgttaaatgcagatgagttaggtttggattttgatggtatgtgtaatgttatcattggattatcagagagcgacttttataaaagcatgaccacctactctgatcatactatctggcaggatgtttacagacccaggcttgttacaggccaggtttatcttaaaattacggtaattcatgacgtactgatcgtctcgtttaaggagaagtaa

SEQ ID NO: 2 - protein, 98 aa, Escherichia coli
MEKRTPHTRLSQVKKLVNAGQVRTTRSALLNADELGLDFDGMCNVIIGLSESDFYKSMTTYSDHTIWQDVYRPRLVTGQVYLKITVIHDVLIVSFKEK

SEQ ID NO: 3 - DNA, 396 bp, Escherichia coli
atgaaatgtccggtttgccaccagggagaaatggtttctggcattaaagatattccatacaccttccgtggacgaaaaacagtattgaaaggtatccacggtttatattgtgtccattgcgaagagagcatcatgaataaagaagagtcagatgctttcatggcgcaagtaaaggcatttcgggcttcggtgaatgccgaaacagtggcacctgaatttatagtgaaggttcgaaaaaagctctctcttacccaaaaagaggcaagcgaaatttttgggggaggtgtaaatgcgttttcgcgttacgaaaaaggcaatgcccaacctcatccttccacaatcaaacttttacgtgttctggataagcatccagaactattgaatgaaatccgttaa

SEQ ID NO: 4 - protein, 131 aa, Escherichia coli
MKCPVCHQGEMVSGIKDIPYTFRGRKTVLKGIHGLYCVHCEESIMNKEESDAFMAQVKAFRASVNAETVAPEFIVKVRKKLSLTQKEASEIFGGGVNAFSRYEKGNAQPHPSTIKLLRVLDKHPELLNEIR

SEQ ID NO: 5 - DNA, 37 bp, Artificial Sequence
  Primer mqsR-Fw
tttttttttcatatggaaaaacgcacaccacatacac

SEQ ID NO: 6 - DNA, 36 bp, Artificial Sequence
  Primer mqsR-Rv
tttgaattcttacttctccttaaacgagacgatcag

SEQ ID NO: 7 - DNA, 30 bp, Artificial Sequence
  Primer ygiT-Fw
tttttttttcatatgaaatgtccggtttgc

SEQ ID NO: 8 - DNA, 40 bp, Artificial Sequence
  Primer ygiT-Rv
tttgaattcttaacggatttcattcaatagttctggatgc

SEQ ID NO: 9 - DNA, 22 bp, Artificial Sequence
  Primer RT-proF
tgcctgactccagcttccctta

SEQ ID NO: 10 - DNA, 31 bp, Artificial Sequence
  Primer RT-proR
ttaacggatttcattcaatagttctggatgc

SEQ ID NO: 11 - DNA, 20 bp, Artificial Sequence
  Primer RT-Fw
acgcacaccacatacacgtt

SEQ ID NO: 12 - DNA, 20 bp, Artificial Sequence
  Primer RT-Rv
gcgaaaacgcatttacacct

SEQ ID NO: 13 - DNA, 31 bp, Artificial Sequence; identical to SEQ ID NO: 10
  Primer YT-Rv

SEQ ID NO: 14 - DNA, 22 bp, Artificial Sequence
  Primer PX-RT
tgtatgtggtgtgcgtttttcc

SEQ ID NO: 15 - DNA, 20 bp, Artificial Sequence
  Primer PX-F1
ttgccaccgtaactgttttc

SEQ ID NO: 16 - DNA, 22 bp, Artificial Sequence
  Primer PX-F2
tgtaacccagtgcatcataaac

SEQ ID NO: 17 - DNA, 21 bp, Artificial Sequence
  Primer PX-F3
gccaacaccgtcgccgttaga

SEQ ID NO: 18 - DNA, 22 bp, Artificial Sequence
  Primer PX-F4
ttttttaccgttgccaagaggt

SEQ ID NO: 19 - DNA, 21 bp, Artificial Sequence
  Primer PX-F5
tggcgaagccgctggtgtttg

SEQ ID NO: 20 - DNA, 20 bp, Artificial Sequence
  Primer PX-F6
gcccacttcaaagtagttca

SEQ ID NO: 21 - DNA, 20 bp, Artificial Sequence
  Primer PX-F7
catgtcgccattgccaccgt

SEQ ID NO: 22 - DNA, 20 bp, Artificial Sequence
  Primer PX-F8
cgaaagaaccaacgtcagcg

SEQ ID NO: 23 - DNA, 20 bp, Artificial Sequence
  Primer PX-F9
ggtagcaacgccgccaacac

SEQ ID NO: 24 - DNA, 19 bp, Artificial Sequence
  Primer PX-F10
gttacgggtttcaccgtag

SEQ ID NO: 25 - DNA, 20 bp, Artificial Sequence
  Primer PX-F11
gcgcaactaacagaacgtct

SEQ ID NO: 26 - DNA, 17 bp, Artificial Sequence
  Primer PX-F12
ttcggcatttaacaaag

SEQ ID NO: 27 - DNA, 21 bp, Artificial Sequence
  Primer PX-A
cagtgtaccaggtgttatctt

SEQ ID NO: 28 - DNA, 20 bp, Artificial Sequence
  Primer Px-Lp
agctgatcgattttagcgtt

SEQ ID NO: 29 - DNA, 20 bp, Artificial Sequence
  Primer B3
agcacacccaccccgtttac

SEQ ID NO: 30 - DNA, 20 bp, Artificial Sequence
  Primer J
ggttcaagatacctagagac

SEQ ID NO: 31 - DNA, 20 bp, Artificial Sequence
  Primer D2
tctctatttatctgaccgcg

SEQ ID NO: 32 - DNA, 20 bp, Artificial Sequence
  Primer E2
tacaggttactttgtaagcc

SEQ ID NO: 33 - DNA, 31 bp, Artificial Sequence
  Palindrome 1F
cccctaactaaccttttaggtgcttttcccc

SEQ ID NO: 34 - DNA, 31 bp, Artificial Sequence
  Palindrome 1R
ggggaaaagcacctaaaaggttagttagggg

SEQ ID NO: 35 - DNA, 27 bp, Artificial Sequence
  Palindrome 2F
cccaattaaccttttaggttataaccc

SEQ ID NO: 36 - DNA, 27 bp, Artificial Sequence
  Palindrome 2R
gggttataacctaaaaggttaattggg

(19) United States
(12) Patent Application Publication
(10) Pub. No.: US 2011/0217282 A1
(43) Pub. Date: Sep. 8, 2011

(54) Novel Toxin-Antitoxin System

(75) Inventors: Masayori Inouye, New Brunswick, NJ (US); Yoshihiro Yamaguchi, Somerset, NJ (US)

(73) Assignee: UNIVERSITY OF MEDICINE AND DENTISTRY OF NEW JERSEY, Somerset, NJ (US)

(21) Appl. No.: 13/059,893

(22) PCT Filed: Aug. 20, 2009
(86) PCT No.: PCT/US09/54503

§ 371 Date: May 13, 2011

Related U.S. Application Data
(60) Provisional application No. 61/189,639, filed on Aug. 20, 2008.

(51) Int. Cl. A61K 38/46; C12N 9/16; C12P 21/00; A61P 35/00; A61P 31/04; A61P 31/12; C12P 19/34

(52) U.S. Cl. 424/94.6; 435/196; 435/69.1; 435/91.3

(57) ABSTRACT

Disclosed in certain embodiments is a method of inhibiting cell function comprising inducing the expression of a mRNA interferase that cleaves mRNA at GCU.

### CLAIM OF PRIORITY

This application claims priority to U.S. Provisional Application No. 61/189,639, filed Aug. 20, 2008, the disclosure of which is hereby incorporated by reference in its entirety.

### SEQUENCE LISTINGS

Sequence listings in written and computer readable form are submitted herewith. The information recorded in computer readable form is identical to the written sequence listings.

### BACKGROUND OF THE INVENTION

It has been reported that quorum sensing is involved in biofilm formation (1-4). MqsR expression was found to be induced eightfold in biofilms (5) and also by the quorum sensing signal autoinducer-2 (AI-2), which is a species-nonspecific signaling molecule produced by both gram-negative and gram-positive bacteria, including E. coli (6). It was reported that induction of MqsR activates a two-component system, the qseBqseC operon, which is known to play an important role in biofilm formation (6). Thus, it has been proposed that MqsR (98 amino acid residues) is a regulator of biofilm formation since it activates qseB, which controls the flhDC expression required for motility and biofilm formation in E. coli (6). However, the cellular function of MqsR has remained unknown.

Interestingly, all free-living bacteria examined to date contain a number of suicide or toxin genes in their genomes (7,8). Many of these toxins are co-transcribed with their cognate antitoxins in an operon (termed as toxin-antitoxin or TA operon), and form a stable complex in the cell so that their toxicity is subdued under normal growth conditions (9-11). However, the stability of antitoxins is substantially lower than that of their cognate toxins so that any stress causing cellular damage or growth inhibition that induces proteases alters the balance between toxin and antitoxin, leading to toxin release in the cell.

To date, sixteen (24) TA systems have been reported on the E. coli genome, including relB-relE (12,13), chpBI-chpBK (14), mazE-mazF (15-17), yefM-yoeB (18,19), dinJ-yafQ (20,21), hipB-hipA, hicA-hicB (25,26), prlF-yhaV (27) and ybaJ-hha (28). Interestingly, all of these TA operons appear to use similar modes of regulation; the formation of complexes between antitoxins and their cognate toxins to neutralize toxin activity and the ability of TA complexes to autoregulate their expression. The cellular targets of some toxins have been identified: CcdB directly interacts with gyrase A and blocks DNA replication (29,30); RelE, which by itself has no endoribonuclease activity, appears to act as a ribosome-associating factor that promotes mRNA cleavage at the ribosome A-site (12,31,32). PemK (33), ChpBK (14) and MazF (34) are unique among toxins, since they target cellular mRNAs for degradation by functioning as sequence-specific endoribonucleases to effectively inhibit protein synthesis and thereby cell growth.

MazF, ChpBK and PemK have been characterized as sequence-specific endoribonucleases, which cleave mRNA at the ACA, ACY (Y is U, A, or G) and UAH (H is C, A, or U) sequences, respectively. They are completely different from other known endoribonucleases such as RNases E, A, and T1, as these toxins function as protein synthesis inhibitors by interfering with the function of cellular mRNAs. It is well known that small RNAs, such as micRNA (mRNA-interfering-complementary RNA) (37), miRNA (38), and siRNA (39), interfere with the function of specific RNAs. These small RNAs bind to specific mRNAs to inhibit their expression. Ribozymes also act on their target RNAs specifically and interfere with their function (40). Therefore, MazF, ChpBK and PemK homologues form a novel endoribonuclease family which exhibits a new mRNA-interfering mechanism by cleaving mRNAs at specific sequences. Thus, they have been termed “mRNA interferases” (2).

All references described herein are incorporated by reference in their entireties for al purposes.

### OBJECTS AND SUMMARY OF THE INVENTION

It has been discovered on the E. coli genome that the MqsR gene is co-transcribed with a downstream gene, YgiT. These two genes appear to function as a TA system, as their size is small (98 residues for MqsR and 131 residues for YgiT) and their respective open reading frames are separated by one base-pair. As disclosed herein, MqsR/YgiT is a new E. coli TA system consisting of a toxin, MqsR and an antitoxin, YgiT. Moreover, as disclosed herein, MqsR is a novel mRNA interferase, which does not exhibit homology to MazF. This toxin cleaves RNA at GCU sequences in vivo and in vitro and therefore has implications in cell physiology and biofilm formation as disclosed herein.

As disclosed herein, the MqsR induction is highly toxic, and its toxicity is blocked by co-expression of YgiT and cellular mRNAs are degraded when MqsR is induced. This in-vivo result was substantiated in vitro using purified MqsR. E. coli total RNA was incubated with MqsR for 30 min at 37° C., clearly indicating that purified MqsR cleaves RNA. Importantly this endoribonuclease activity was completely inhibited when its presumed antitoxin, YgiT was added in the reaction mixture. With use of 3.5-kbase phage MS2 RNA, we have identified the major cleavage sites by this toxin. Thus, it appears to be a highly sequence-specific mRNA interferase, that recognizes a triplet sequence, GCU.

This sequence may be either underrepresented or overrepresented in some genes, and the genes may be associated with quarum sensing and/or biofilm formation.

Accordingly, this invention relates to a new TA system, MqsR YgiT in E. coli. The induction of MqsR was highly toxic in E. coli and caused a degradation of mRNA in vivo. Purified MqsR showed endoribonuclease activity and YgiT neutralized the activity in vitro. MqsR cleaves MS2 phage RNA at GCU.

The invention can be used in single-protein production in prokaryotic and eukaryotic cells, such as E. coli and mammalian cells. It also has applications in gene therapy by using the MqsR/YgiT system to treat various human diseases such as cancer, bacterial infection and viral infection including AIDS. The invention can be used as an RNA restriction enzyme for RNA structural study.

In certain embodiments, the invention is directed to a method of inhibiting cell function comprising inducing the expression of a mRNA interferase that cleaves mRNA at GCx, wherein x is A, C, G, or U. The mRNA interferase can be ribosome-independent and is preferably MqsR or a homolog thereof. In alternative embodiments, the induction is capable of being inhibited by an antitoxin, e.g., YgiT. The cell can be, e.g., E. coli or Homo sapiens.

In embodiments disclosed herein, the inhibition of mRNA interferase can be either in-vitro or in-vivo.

In certain embodiments, the invention is directed to a method of inhibiting cell function comprising inducing the expression of MqsR or a homolog thereof.

In certain embodiments, the invention is directed to a plasmid comprising a gene encoding MqsR or a homolog thereof. The expression of MqsR can be induced, e.g., with IPTG and can be, e.g., a pET28a plasmid. In alternative embodiments, the gene has a sequence according to SEQ ID NO: 1.

In certain embodiments, the invention is directed to a plasmid comprising a gene encoding YgiT or a homolog thereof. The expression of YgiT can be induced, e.g., with arabinose and can be, e.g., a pBAD24 plasmid. In alternative embodiments, the gene has a sequence according to SEQ ID NO: 3.

In certain embodiments, the invention is directed to a plasmid comprising: a) a gene encoding MqsR or a homolog thereof; and b) a gene encoding YgiT or a homolog thereof. In alternative embodiments, the gene encoding MqsR has a sequence according to SEQ ID NO: 1 and the gene encoding YgiT has a sequence according to SEQ ID NO: 3.

In certain embodiments, the invention is directed to a cell (e.g., E. coli or Homo sapiens) transformed with one or more plasmids disclosed herein.

In certain embodiments, the invention is directed to a method of inhibiting MqsR endoribonuclease activity comprising contacting MqsR with YgiT. In alternative embodiments, the method comprises pre-incubating MqsR with YgiT.

In certain embodiments, the invention is directed to the use of YgiT as an antitoxin for MqsR.

In certain embodiments, the invention is directed to a method of inhibiting cell lysis of E. coli comprising inactivating MqsR. In alternative embodiments, the MqsR is inactivated by YgiT.

In certain embodiments, the invention is directed to an isolated YgiT polypeptide having an amino acid sequence according to SEQ ID NO: 4. In alternative embodiments, the polypeptide has an amino acid sequence which has 90% homology with this amino acid sequence and has antitoxin activity.

In certain embodiments, the invention is directed to an isolated YgiT polynucleotide having a DNA sequence according to SEQ ID NO: 3. In alternative embodiments, the polynucleotide has a DNA sequence which has 90% homology with this DNA sequence and encodes a polypeptide having antitoxin activity.

In certain embodiments, the invention is directed to a complex comprising MqsR and YgiT, or homologs thereof. In alternative embodiments, the complex comprises a polypeptide according to SEQ ID NO: 2 and a polypeptide according to SEQ ID NO: 4.

In certain embodiments, the invention is directed to a method of producing a polypeptide having endoribonuclease activity comprising: a) transforming a cell by introducing a polynucleotide encoding MqsR into the cell, and b) culturing the transformed cell.

In certain embodiments, the invention is directed to a method of producing a polypeptide having antitoxin activity comprising: a) transforming a cell by introducing a polynucleotide encoding YgiT into the cell, and b) culturing the transformed cell.

In certain embodiments, the invention is directed to a method of cleaving mRNA comprising contacting an mRNA interferase with mRNA wherein the mRNA interferase is not homologous to MazF. In alternative embodiments, the mRNA is cleaved at GCx, wherein x is A, C, G, or U.

In certain embodiments, the invention is directed to a method of altering cell function comprising manipulating the expression of one or both of MqsR and YgiT.

In certain embodiments, the invention is directed to a method of treating a patient with a disease comprising administering to the patient a mRNA interferase that cleaves mRNA at GCx, wherein x is A, C, G, or U. The disease can be, e.g., cancer, bacterial infection or viral infection. The viral infection can be, e.g., caused by HIV or a retrovirus.

In certain embodiments, the invention is directed to a method of treating a patient with a disease comprising administering to the patient a gene encoding a mRNA interferase that cleaves mRNA at GCx, wherein x is A, C, G, or U. The disease can be, e.g., cancer, bacterial infection or viral infection. The viral infection can be an infection caused by a virus having a single-stranded RNA genome, e.g., HIV or a retrovirus.

In certain embodiments, the invention is directed to a primer according to any one of SEQ ID NOs 5-36

### BRIEF DESCRIPTION OF THE DRAWINGS

FIG. 1 shows a gene map of the MqsR-YgiT operon on the E. coli chromosome. A. Arrows indicate the direction and size of the respective genes: qseC, qseB, ygiW, ygiV, mqsR, ygiT, ygiS and parC. The MqsRYgiT promoter sequence is also shown and the palindromic sequences (1 and 2) are boxed. The bent arrow represents the transcription initiation site of the MqsR-YgiT operon. The −10 and −35 regions of the MqsRYgiT promoter are shown in bold and the Shine-Dalgarno sequence, GGAGG is boxed. Underlined DNA sequences were used in EMSA assay as shown in FIG. 5. B. RT-PCR analysis of the MqsRYgiT operon. cDNA was synthesized with reverse transcriptase using total RNA from E. coli BL21 strain grown at 37° C. to an O.D.₆₀₀ of 0.8. Using the cDNA product as template, PCR was carried out with RT-Fw and RT-Rv primers. Lane 1, 100-bp DNA ladder (Genscript); lanes 2 and 4, cDNA and genomic DNA was used as template for PCR, respectively; and lane 3, PCR products without using reverse transcriptase. C. The transcriptional start site of MqsRYgiT. Primer extension analysis was carried out using the same RNA described in the legend to FIG. 1B and the PX-RT primer. G, A, T and C (lanes 1 to 4) comprise the sequence ladders using pCR®2.1-Topo®-MqsRYgiT and the same primer. The transcriptional start site is indicated by letter+1.

FIG. 2 shows the effect of MqsR induction on protein and DNA synthesis and mRNA stability. A. E. coli BL21 transformed with pET-MqsR and pBAD-YgiT was streaked on M9 (glycerol, CAA) plates with 0.1 mM IPTG, 0.2% arabinose, 0.1 mM IPTG plus 0.2% arabinose or without both inducers. The plates were incubated at 37° C. for 18 h. B. Growth curves of E. coli BL21 cells harboring pBAD-MqsR. The cells were cultured in M9-glycerol liquid medium at 37° C. in the presence (closed circles) or absence (open circles) of 0.2% arabinose. C. Effect of MqsR on [³⁵S]methionine incorporation in vivo. At the different time intervals indicated, 0.4 ml of the culture was taken into a test tube containing 30 μCi of [³⁵S] methionine, and the mixture was incubated for 30 second at 37° C. After the incubation, 50 μl of the reaction mixture was applied to a filter paper disk (Whatman 3 mm, 2.3 cm diameter). The filter paper disks were treated in 10% trichloroacetic acid solution as described previously (34). The radioactivity on filter was determined with a liquid scintillation counter. D. SDS-PAGE analysis of the products from C. Four hundred micro litter of the reaction mixture at the time points indicated was put into a chilled test tube containing 100 μg/ml non-radioactive methionine, and cells were collected by centrifugation. The pellets were dissolved in 40 μl SDS-PAGE loading buffer. The samples were incubated in a boiling water bath for 10 min. After removing insoluble materials by centrifugation, the supernatant fraction (12.5 μl) was applied to 15% SDS-PAGE gel. E. Effect of MqsR on [³H]thymidine incorporation in vivo. E. coli BL21 cells harboring pBAD-MqsR were grown at 37° C. When the O.D.₆₀₀ value of the culture reached 0.3, MqsR were induced with arabinose (0.2%). At the different time intervals indicated, 0.4 ml of the culture was taken into a test tube and incubated with 10 μCi of [³H]thymidine plus 30 μg non-radioactive thymidine. Then, the mixture was incubated for 30 seconds at 37° C. After the incubation, the incorporated radioactivity into the cells were determined as described previously (34). F. Effect of MqsR on cellular mRNA stability. Total RNA was extracted from E. coli BL21 cells harboring pBAD-MqsR at various time points as indicated after the addition of arabinose (0.2%) and subjected to Northern blotting with labeled ompA, ompF and lpp as probes, respectively. Before transferring RNA onto membrane, the gel was stained with ethidium bromide to detect 23S rRNA and 16S rRNA.

FIG. 3 shows a primer extension analysis of MqsR cleavage sites in the ompF mRNA in vivo. Total RNA was prepared from E. coli BL21 cells harboring pBAD-MqsR at indicated time points before and after the induction of MqsR. The sequence ladders were obtained with pCR®2.1-Topo®-ompF as template (34). The sequences around the cleavage sites were indicated at the bottom and the cleavage sites are indicated by arrows.

FIG. 4 shows mRNA interferase activity of MqsR in vitro. A. Effect of H-MqsR on protein synthesis in a cell-free system. MazG protein synthesis was carried out using E. coli T7 S30 extract system for circular DNA (Promega) with peT11a-mazG. Lane 1, without H-MqsR; lanes 2 to 6, 5, 10, 20, 40 and 80 nM H-MqsR were added, respectively; lane 7, 80 nM H-MqsR plus 40 nM YgiT-H; and lane 8, 40 nM YgiT-H was added. B. mRNA interferase activity of purified H-MqsR in vitro. MS2 phage RNA (0.8 μg) was incubated with H-MqsR at 37° C. for 10 min in 10 mM Tris-HCl (pH 8.0) containing 1 mM DTT. The products were separated on a 1.2% agarose gel. The gel was stained with ethidium bromide.

FIG. 5 shows the binding of MqsR, MqsRYgiT and YgiT to the palindromic sequences in the MqsRYgiT 5′-UTR region. The electrophoretic mobility shift assay (EMSA) was carried out with 5′-end-labeled palindrome 1 (lanes 1 to 6) and 2 (lanes 7 to 12) DNA fragments (see FIG. 1A), which were incubated with different concentrations of proteins as described in Experimental Procedures. Lanes 1 to 6 and lanes 7 to 12 represent 0, 5, 10, 20, 40, and 80 nM of H-MqsR (A), YgiT-H (B) and H-MqsRYgiT (C), respectively.

FIG. 6 shows a general genetic context of a TA loci.

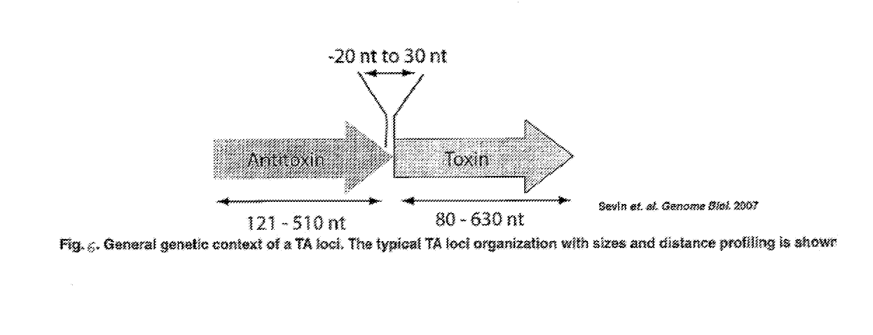

FIG. 7 shows a model of regulation of biofilm formation by MqsR in E. coli.

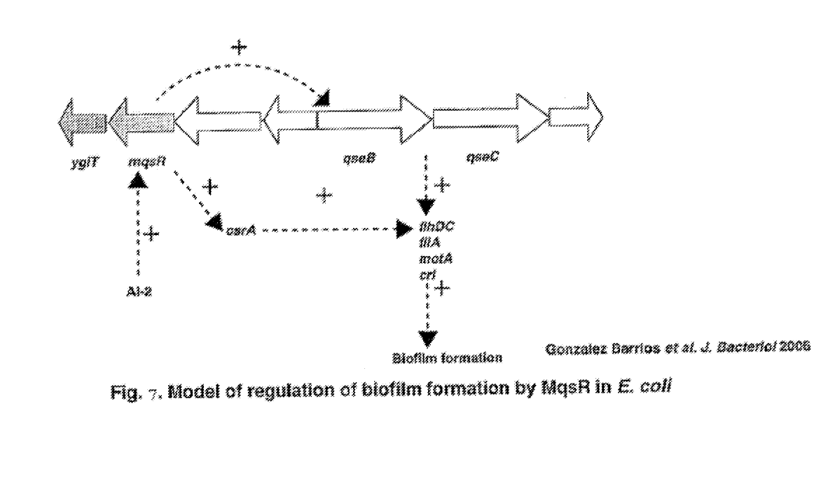

FIG. 8 shows the effect of MqsR on mRNA stability and protein and DNA synthesis.

FIG. 9 shows the effect of His-MqsR on protein synthesis in a prokaryotic cell-free system.

FIG. 10 shows cleavage of total RNA and MS2 phage RNA by purified His-MqsR.

FIG. 11 shows primer extension analysis of an MqsR cleavage site in the MS2 RNA in vitro.

### EXPERIMENTAL PROCEDURES

The invention is further described by the following non-limiting experimental procedures.

Toxicity of MqsR in E. coli.

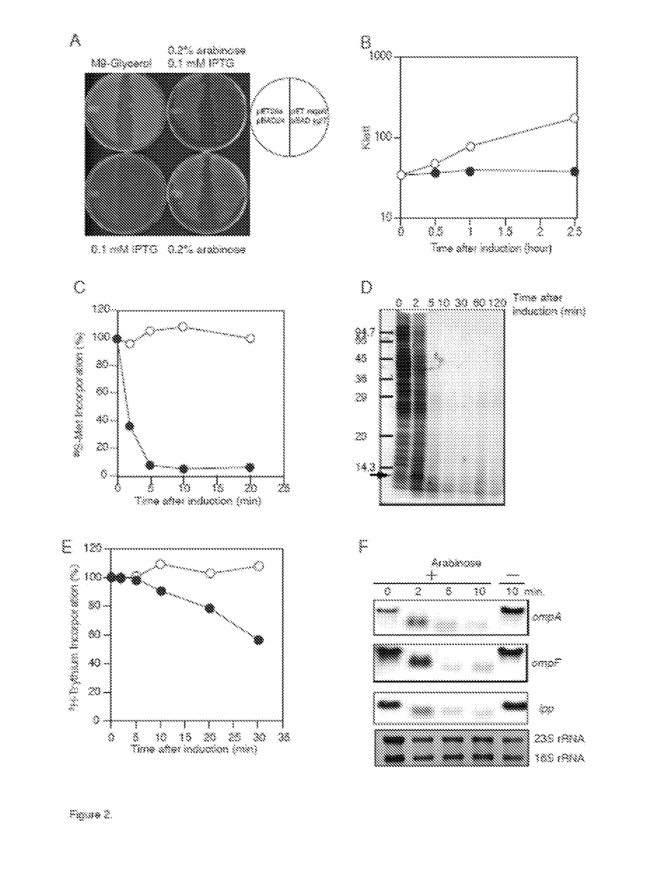

E. coli BL21 cells were transformed with pET-MqsR and pBAD-YgiT or pBAD and pET plasmids. The cells were spread on glycerol-M9-casamino acids agar plates with and without inducers [arabinose (0.2%) and IPTG (0.1 mM)] and these plates were incubated at 37° C. for 24 h. as shown in FIG. 2A. FIG. 2B shows growth curves of E. coli BL21 harboring pBAD-MqsR plasmid in M9 (glycerol, CAA) liquid medium at 37 C in the presence (closed circles) and the absence (open circles) of 0.2% arabinose. Cell growth was measured by A (absorbance) at 600 nm.

Effect of MqsR on mRNA Stability and Protein and DNA Synthesis

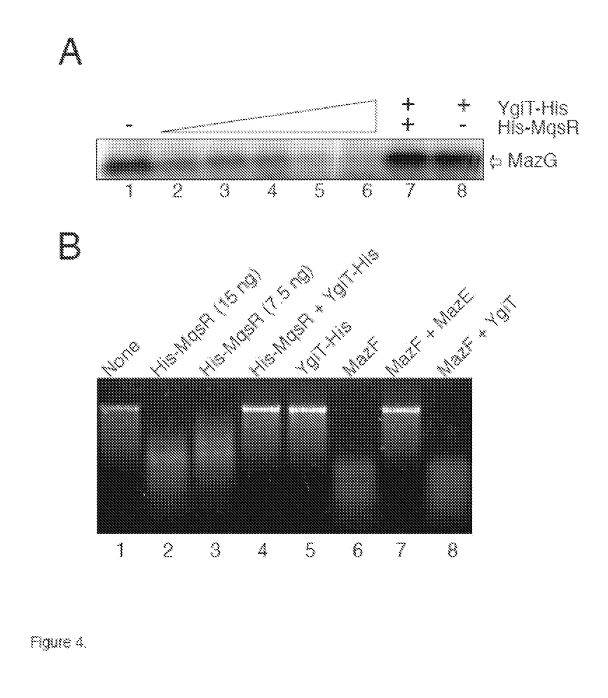

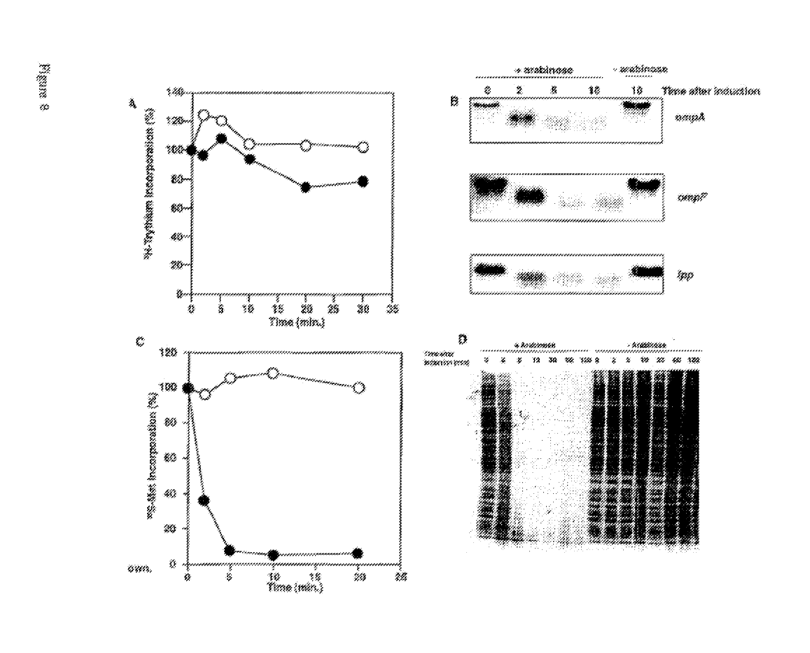

Total cellular RNA was extracted from E. coli BL21 cells containing pBAD-MqsR at various time points as indicated after the addition of arabinose and subjected to Northern blot analysis using radiolabeled lpp, ompF, and ompA ORF DNA as probes. FIG. 4A shows the effect of MqsR on [3H]dTTP incorporation in vivo. FIG. 4B shows the effect of MqsR on Cellular mRNAs in vivo. ³⁵S-methionine incorporation into E. coli BL21 cells containing pBAD-MqsR was measured at various time points as indicated after MqsR induction. FIG. 4C shows the effect of MqsR on ³⁵S-methionine incorporation in vivo. The same cultures in (4C) were used to show the SDS-PAGE analysis of in vivo protein synthesis after the induction of MqsR, as shown in FIG. 8D.

### Effect of His-MqsR on Protein Synthesis in a Prokaryotic Cell-Free System.

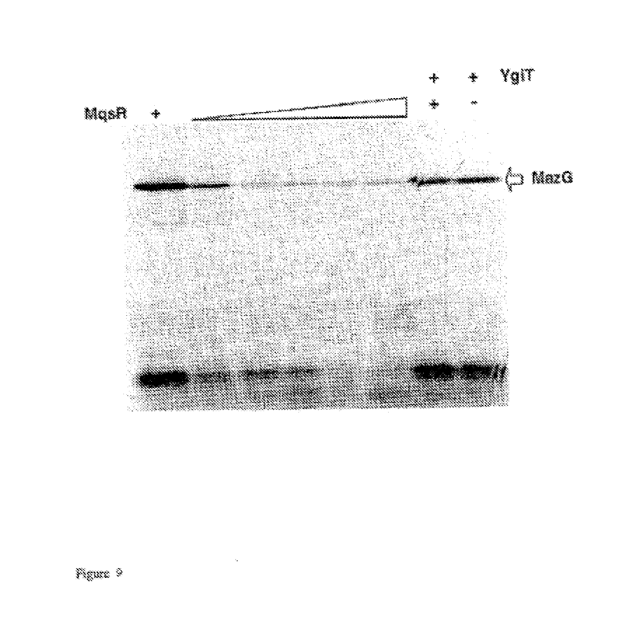

MazG protein synthesis was performed in the E. coli T7 S30 extract system (Promega) with pET-11a-MazG as template. The results are shown in FIG. 9.

### Cleavage of Total RNA and MS2 Phase RNA by Purified His-MqsR.

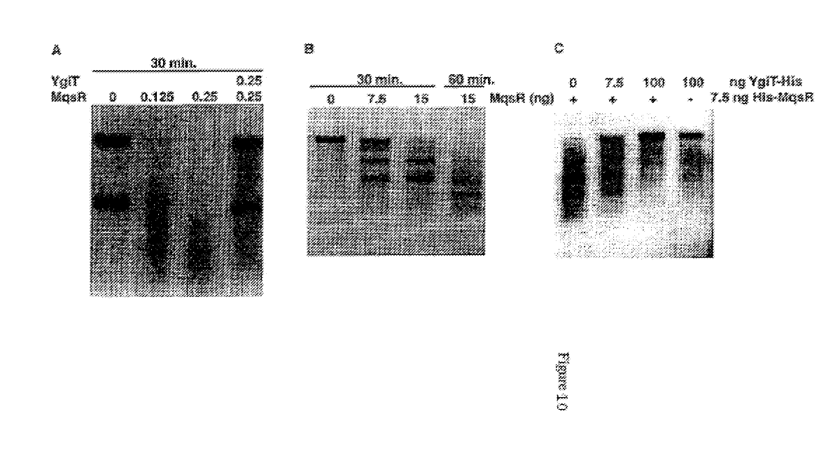

E. coli total RNA was incubated with purified His-tagged MqsR for 30 min at 37° C. In the last lane, purified YgiT was added. RNA was analyzed in 1.2% TBE agarose gel and the gel was stained with ethidium bromide (EtBr), as shown in FIG. 10A.

Cleavage of MS2 ssRNA and its Inhibition by YgiT.

MS2 ssRNA (0.8 μg; 3569 bases; Roche) was digested by His-MqsR in 20 at 37° C. His-MqsR was preincubated with purified YgiT for 10 min on ice and then further incubated with MS2 RNA for 30 min. Denatured products in urea were separated on 1.2% TBE native agarose gel. The gel was stained with EtBr. The results are shown in FIGS. 10B and 10C.

### Primer Extension Analysis of a MqsR Cleavage Site in the MS2 RNA In Vitro.

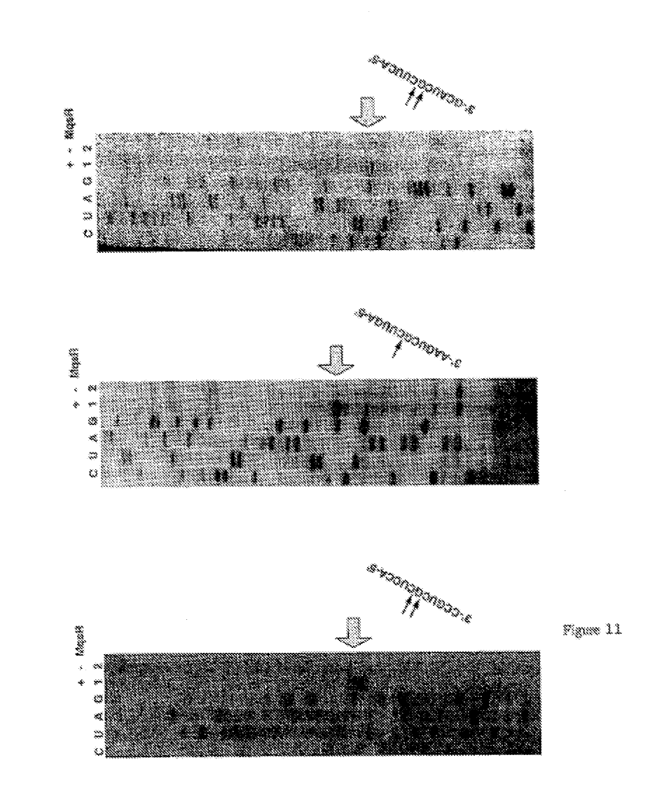

In vitro cleavage of the MS2 RNA with His-MqsR. Lane 1, MS2 RNA with His-MqsR; lane 2, represents a control reaction in which no proteins were added; Cleavage sites are indicated by red arrows on the RNA sequence and were determined using the RNA ladder shown on the left. The results are shown in FIG. 11.

### Further Detailed Experimental Procedures

Bacterial strains and plasmids—E. coli BL21(DE3) and C43 were used. Both MqsR and YgiT genes in the MqsRYgiT operon, were separately amplified by PCR using the E. coli genomic DNA as template and first cloned into pET28a (Novagen). The MqsRYgiT operon was also amplified by PCR with MqsR-Fw and YgiT-Rv primers using the E. coli genomic DNA as template and cloned into pET28a to express the MqsR-YgiT complex. Subsequently, the MqsR and YgiT genes were separately cloned into pBAD24 creating pBAD-MqsR and pBAD-YgiT, respectively. The promoter region of MqsRYgiT was amplified by PCR with RT-proF and RT-proR primers and cloned into pCR®2.1-Topo® vector (invitrogen).

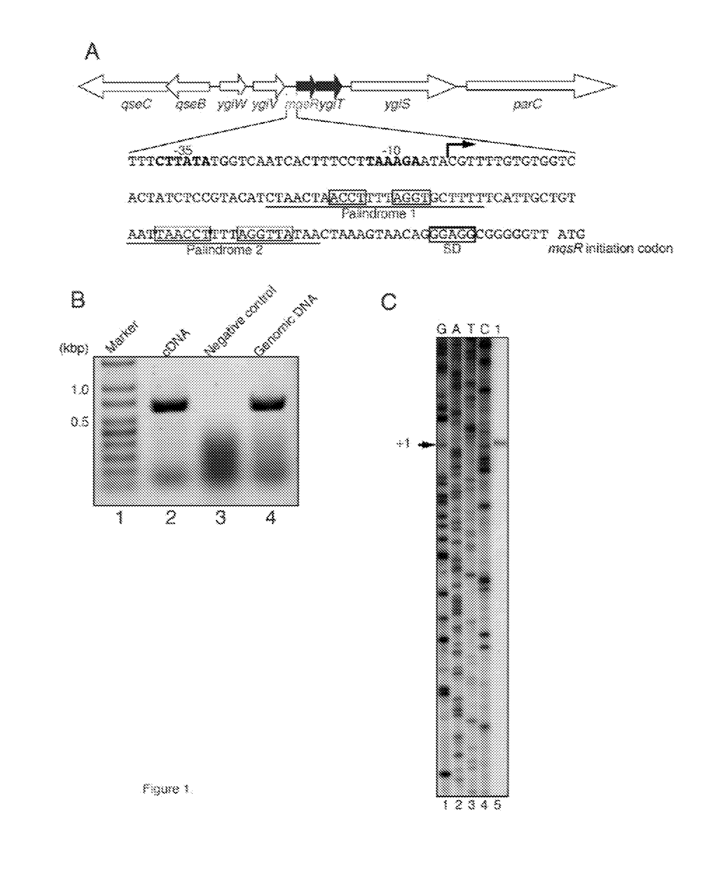

Assay of in vivo DNA and protein synthesis—E. coli BL21(DE3) cells harboring pBAD-MqsR were grown in M9 medium with 0.5% glycerol (no glucose) and 1 mM of each amino acids except for methionine. When the O.D.₆₀₀ value of the culture reached 0.3, arabinose was added to a final concentration of 0.2% to induce MqsR. Aliquots of the cell cultures (0.4 ml) were taken at time intervals as indicated in FIG. 2 and mixed with 30 μCi [³⁵S]-methionine or 10 μCi [³H]thymidine plus 80 μg of non-radioactive methionine and 30 μg of non-radioactive thymidine, respectively). After incubation at 37° C. for 30 seconds, the rates of protein and DNA synthesis were determined as described previously (34). For SDS-PAGE analysis of the total cellular protein synthesis, 400 μl samples were removed from the reaction mixture containing [³⁵S]-methionine at the time intervals indicated in FIG. 1F and transferred to chilled test tubes containing 100 μl of 100 μg/ml non-radioactive methionine solution. Cell pellets were collected by centrifugation, resuspended in 40 μl of Laemmli buffer and subjected to SDS-PAGE followed by autoradiography.

RNA isolation and Northern blotting analysis—E. coli BL21(DE3) cells containing pBAD-MqsR were grown at 37° C. in M9 medium with 0.2% glycerol (no glucose). When the O.D.₆₀₀ value reached 0.4, arabinose was added to a final concentration of 0.2%. The samples were taken at different intervals as indicated in FIG. 2. Total RNA was isolated using the hot-phenol method as described previously (35). Northern blot analysis was carried out as described previously (36).

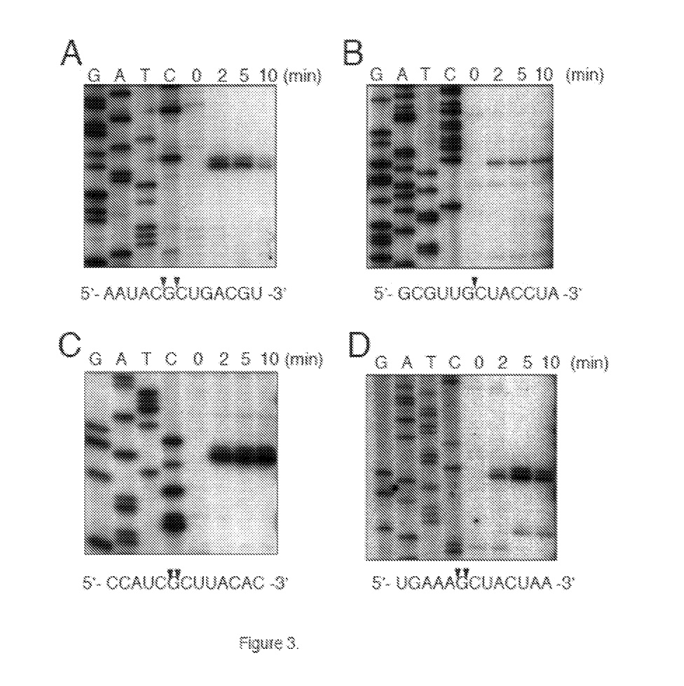

Primer extension analysis in vivo—For primer extension analysis of mRNA cleavage sites in vivo, total RNAs were extracted from the E. coli BL21(DE3) cells containing pBAD-MqsR at different time points after MqsR induction as indicated in FIG. 3. Primer extension was carried out at 47° C. for 1 h with 10 units of AMV-reverse transcriptase (AMV-RT) (Roche) using 15 μg of total RNA and 1 μmol of the primers (Table 1) labeled with T4 polynucleotide kinase (Takara Bio) with [γ-³²P]-ATP. The reaction was stopped by addition of 12 μl of sequencing loading buffer (95% formaldehyde, 20 mM EDTA, 0.05% bromophenol blue and 0.05% xylene cyanol) and heated at 95° C. for 2 min and then placed on ice. The products were analyzed on a 6% polyacrylamide containing 8 M urea gel with a sequencing ladder made with the same primer.

Protein purification—To purify N-terminal histidine-tagged MqsR (H-MqsR) and C-terminal histidine-tagged YgiT (YgiT-H), pET-MqsRYgiT and pET-YgiT were introduced into E. coli BL21(DE3). The expression of H-MqsRYgiT complex and YgiT-H was induced with 1 mM isopropyl-b-D-1-thiogalactoside (IPTG) for 3 h, respectively. The H-MqsRYgiT complex and YgiT-H were purified with Ni-NTA agarose (Qiagen) following the manufacture's protocol. Subsequently, the H-MqsRYgiT complex was denatured with 6M guanidine HCl. Denatured H-MqsR was then purified with Ni-NTA agarose and refolding of H-MqsR was carried out by stepwise dialysis as previously described for MazF (16).

Assay of protein synthesis in vitro—Cell-free protein synthesis was performed with an E. coli T7 S30 Extract System for Circular DNA (Promega). The reaction mixture was prepared as described in the manufacture's protocol. Then, different amounts of H-MqsR and YgiT-H were added in a final volume of 29 The reaction was started by the addition of pET11a-mazG plasmid DNA (18,37) and the mixture was incubated for 1 h at 37° C. Proteins were precipitated with acetone and analyzed by 15% SDS-PAGE. The dried gel was analyzed by autoradiography.

mRNA interferase activity of MqsR—MS2 phage RNA (Roche) was incubated with H-MqsR in 10 mM Tris-HCl buffer (pH 8.0) containing 1 mM dithiothreitol (DTT) at 37° C. for 10 min. In order to examine the antitoxin function of YgiT, H-MqsR was preincubated with YgiT-H for 10 min on ice and then further incubated with MS2 RNA for 10 min. After denaturation in urea, the products were separated on 1.2% agarose gel in 0.5×TBE buffer (44.5 mM Tris borate and 1 mM EDTA)(38).

Primer extension analysis in vitro—MS2 RNA was incubated with or without purified H-MqsR in 10 mM Tris-HCl (pH 8.0) containing 1 mM DTT at 37° C. for 15 min and the digested MS2 RNA (0.8 μg) was used for primer extension as described above.

Electrophoretic mobility shift assays (EMSA)—Complementary strands (Table 1) were annealed, and purified to get palindrome 1 and 2 double-stranded DNA, respectively. The double stranded DNA fragments were end-labeled with [g-³²P]ATP by T4 kinase (Takara Bio). The binding reactions were carried out at 4° C. for 30 min in 50 mM Tris-HCl (pH 7.2) buffer containing 50 mM KCl, 5% glycerol, 100 ng poly(dI-dC), labeled DNA fragment and purified proteins. Electrophoresis was performed at 4° C. in TE buffer (10 mM Tris-HCl, 1 mM EDTA, pH 7.2) at 110 V in 5% acrylamide/bisacrylamide (40:1.2) gel. After electrophoresis, the gel was dried and analyzed by autoradiography.(39).

Reverse transcription (RT)-PCR—Total RNA from E. coli was extracted at exponential phase (O.D.₆₀₀ of 0.8) as described above and treated with 100 units of RNase-free DNase I (Promega) in the presence of 0.5 μl (20 units) RNase inhibitor (Roche). The RT reaction was carried out at 47° C. for 1 h using total RNA (20 μg) and the primer YT-Rv (20 μmol) with 10 units AMV-RT (Roche). PCR was carried out using the synthesized cDNA as template with RT-Fw and RT-Rv primers (Table 1).

### Results

The MqsR and YgiT genes are in an operon—The location of the MqsRYgiT operon at 68 min on the E. coli K-12 chromosome is shown in FIG. 1A. MqsR is a 98-residue protein and there is a predicted Shine-Dalgarno sequence (GGAGG) eight bases upstream of the initiation codon for its ORF (open reading frame) (boxed in FIG. 1A). The downstream YgiT is a 131-residue protein and the initiation codon of YgiT is one base downstream of the translational stop codon of MqsR. In order to determine whether MqsRYgiT is transcribed as an operon, reverse transcription polymerase chain reaction (RT-PCR) was carried out using total RNA extracted from E. coli BL21(DE3). The cDNA was synthesized from total RNA using YT-Rv primer (Table 1), which is located 31-bp upstream of the YgiT stop codon, as described in Experimental Procedures. As shown in FIG. 1B; lane 2, the band was detected at the position of approximately 600 bp PCR using RT-Fw and RT-Rv (Table 1) as primers. When the E. coli genomic DNA was used as template for PCR with the same primers, the expected 576-bp band was detected (FIG. 1B; lane 3). This band was not detected in the reaction carried out without the addition of reverse transcriptase (FIG. 1B; lane 2). These results demonstrate that the MqsR gene is co-transcribed with the downstream gene, YgiT. In order to identify the transcription initiation site, we performed primer extension using the same RNA described above with PX-RT primer. The primer is located 2 bp downstream of the initiation codon of the MqsR gene. As shown in FIG. 1C, the transcription start site is located 109 bp upstream of the MqsR start codon, as indicated by the arrow. Thus, we identified the −10 region and the −35 region, a typical RNA polymerase promoter, in the upstream region of the transcription initiation site as shown in FIG. 1A. No transcription start sites were detected in the region between MqsR and YgiT (data not shown) indicating that there is no independent transcriptional unit for the YgiT gene. Also, there are two palindrome sequences in the 109-base 5′-untranslated region (5′-UTR) as indicated by boxes in FIG. 1A.

The effect of MqsR on cell growth—The MqsR and YgiT genes were cloned into an IPTG inducible pET28a plasmid (Novagen) and an arabinose inducible pBAD24 plasmid (40), respectively. E. coli C43 cells harboring pET-MqsR and pBAD-YgiT could not form colonies on M9-glycerol-casamino acids agar plates in the presence of arabinose (0.2%)(FIG. 2A). However, co-induction of YgiT in the presence of 0.2% arabinose neutralized the toxicity of MqsR leading to the formation of colonies indicating that MqsR is the toxin, while YgiT is the antitoxin for MqsR. We also examined the toxicity of MqsR in a liquid culture (FIG. 2B). When MqsR was induced by the addition of arabinose (0.2%), cell growth was completely inhibited after 30 min.

Next, we examined the effect of MqsR induction on protein synthesis as measured by [³⁵S]methionine incorporation. Within 5 min of MqsR induction, the protein synthesis was almost completely inhibited (FIG. 2C). These samples were analyzed by SDS-PAGE (FIG. 2D). Consistent with the result in FIG. 2C, MqsR completely blocked the incorporation of [³⁵S]methionine into cellular proteins. The strong band present in the 2 min time point (indicated by an arrow) with an apparent molecular weight of 12 kDa is likely MqsR (MW, 11232). These results showed that MqsR is a general inhibitor for the synthesis of all cellular proteins. Indeed, the incorporation of [³H]thymidine was not significantly affected upon MqsR induction (FIG. 2E), indicating that MqsR inhibits protein synthesis but not DNA synthesis. When the cellular mRNAs (ompA, ompF and lpp) of E. coli BL21(DE3) cells carrying pBAD-MqsR were analyzed by Northern blotting at different time points after induction of MqsR by arabinose, the full length mRNAs were observed only at the 0 time point in all cases (FIG. 2F). At 2 min, the full size mRNAs were shortened by a certain length, indicating that all mRNAs tested have a preferential initial cleavage site located near the 5′ end or the 3′ end. The intensity of these bands was significantly reduced after 5 min. These data suggest that MqsR possesses endoribonuclease activity and inhibits protein synthesis through the cleavage of mRNA. It is important to note that 16S and 23S rRNA were very stable in vivo even 10 min after MqsR induction, as no significant changes in their band intensities were observed (FIG. 2F). This was similar to the result seen with MazF mRNA interferase (34). The rRNAs appear to be protected from MqsR cleavage by the ribosomal proteins.

In vivo cleavage of the ompA, ompF and lpp mRNAs by MqsR—Next, we examined the MqsR-mediated cleavage of the ompA, ompF and lpp mRNAs by primer extension experiments. Primer extension analysis of ompA, ompF and lpp using different primers identified distinct bands that appeared 2 min after induction of MqsR corresponding to the specific cleavage sites in each mRNA (Table 2 and FIG. 3 A-D). These bands were not detected at 0 min. From the alignment of all cleavage sequences, the cleavage occurred before or after the G residue of GCU sequences indicating that MqsR cleaves mRNAs at the specific sequence, GCU, in vivo. All of the GCU sequences in the ompF mRNA were cleaved after MqsR induction without exception (Table 2).

The mRNA interferase activity of MqsR in vitro—In order to obtain purified MqsR, N-terminal histidine-tagged MqsR(H-MqsR) was first expressed as the H-MqsRYgiT complex from the E. coli BL21(DE3) cells harboring pET-MqsRYgiT and the complex was purified with Ni-NTA agarose. Then, the purified H-MqsRYgiT complex was denatured using 6 M guanidine HCl. Denatured H-MqsR was re-trapped on Ni-NTA agarose, eluted and refolded by stepwise dialysis (16). C-terminal histidine-tagged YgiT (YgiT-H) was expressed in E. coli and purified as described in Experimental Procedures. The molecular mass of the purified H-MqsR, YgiT-H and H-MqsRYgiT complex were determined to be 26, 32 and 90 kDa by gel filtration, respectively (data not shown). The results suggests that both MqsR and YgiT exist as dimer and that the MqsRYgiT complex likely consists of two MqsR dimers and one YgiT dimer, which is also the case of the MazEMazF complex (16).

We next examined the effect of H-MqsR and H-MqsRYgiT on cell-free protein synthesis using an E. coli T7 S30 extract system (Promega). The synthesis of MazG protein was almost completely inhibited by 40 nM or higher concentration of MqsR (FIG. 4A; lanes 5 and 6). Inhibition of protein synthesis in vitro was observed in the case of MazF (34) and YoeB (18). The YgiT-H and H-MqsRYgiT complex did not inhibit protein synthesis (FIG. 4A; lanes 7 and 8).

To further prove that the in vivo cleavage of ompA, ompF and lpp mRNAs observed above was due to the mRNA interferase activity of MqsR, MS2 phage RNA (3569 bases) was cleaved with purified MqsR-H in vitro. The purified MqsR preparation clearly showed endoribonuclease activity (FIG. 4B, lanes 2 and 3). The endoribonuclease activity was completely inhibited when purified YgiT-H was preincubated with H-MqsR (FIG. 4B, lane 4). Purified YgiT-H by itself had no detectable effect on the mRNA (FIG. 4B, lane 5). The results confirm that YgiT functions as an antitoxin and blocks the MqsR mRNA interferase activity. To confirm that YgiT is the specific inhibitor for MqsR, we examined if YgiT inhibits MazF, which cleaves mRNA at ACA sequence. MazF cleaved MS2 RNA (FIG. 4B, lane 6) and its activity was completely inhibited when it was preincubated with purified MazE, the antidote of MazF (FIG. 4B, lane 7). However, when MazF was incubated with purified YgiT-H, its activity was not inhibited (FIG. 4B, lane 8). This result showed that YgiT specifically inhibits MqsR endoribonuclease activity.

The ability of MqsR to cleave RNA in the absence of ribosomes is distinctly different from RelE or YoeB whose mRNA interferase activities are dependent on ribosomes (12,18,41). The activity of MqsR activity was inhibited by MgCl₂ (data not shown) as described previously for MazF (34).

In vitro cleavage site of MS2 RNA by purified MqsR—The in vitro MqsR activity on MS2 RNA was also analyzed by primer extension. The MS2 RNA was incubated at 37° C. for 10 min with MqsR. The product was used as template for primer extension. MqsR cleaved the MS2 RNA at five cleavage sites and the sequences of all of the cleaved sites were determined to be GCU (Table 2). Taken together, the results of the in vivo and in vitro primer extension experiments (FIG. 3 and Table 2), indicate that MqsR is an mRNA interferase specifically cleaving RNA at GCU sequences.

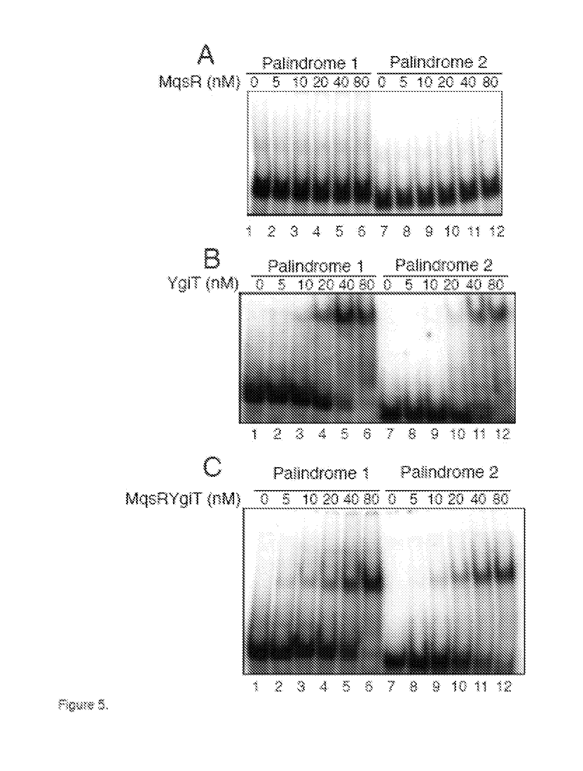

The binding of the MqsRYgiT complex to the MqsRYgiT promoter region—There are palindromic sequences in the promoter regions of many other TA systems including ccdAB (42,43), parDE (44), mazEF (45) and relBE (46). These antitoxins or toxin-antitoxin complexes bind to their cognate palindromic sequence to negatively regulate their own operons. Since there are two palindromic sequences in the 5′-UTR region of the MqsRYgiT operon (FIG. 1A), we next examined if the MqsRYgiT complex was able to bind them. Palindrome 1 and 2 DNA fragments were prepared as described in Experimental Procedures and labeled with [γ-³²P]ATP by T4 kinase. YgiT and the MqsRYgiT complex were mixed with labeled DNA to test their ability to bind the palindrome sequences. YgiT was able to shift the mobility of palindrome 1 and 2 fragments at 10 and 20 nM or higher concentrations (FIG. 5A; lanes 3 to 6 and 10 to 12), respectively. At 5 nM, no shifted bands were observed with either palindrome 1 or 2 fragments. Notably, H-MqsR protein alone could not bind to either palindromic sequence, even at 80 nM concentration (FIG. 5A). However, the addition of MqsR to YgiT enhanced YgiT binding to both palindromic sequences. MqsR was added to YgiT at a molar ratio of 2 to 1. The complex binds to both palindromic sequences stronger as compared to YgiT alone (FIG. 5C; lanes 2 to 6 and 9 to 12, respectively). Under these conditions, positions of the bands representing the palindromic sequences were shifted at 5 and 10 nM MqsRYgiT complex for palindrome 1 and 2 fragments, respectively. The result suggests that both YgiT and MqsRYgiT complex bind to the palindromic sequences to negatively regulate the MqsRYgiT operon like other TA systems.

### Discussion

As disclosed herein, we demonstrated that the MqsR and YgiT genes on the E. coli chromosome are co-transcribed and MqsR-YgiT is a new toxin-antitoxin system. In contrast to most of other TA systems, the first gene in the operon encodes the toxin, MqsR, and the second gene encodes the antitoxin, YgiT. Although MqsR has no homology to the well-characterized mRNA interferase MazF, which specifically cleaves at ACA sequences in mRNAs (29), MqsR was found to be an mRNA interferase that cleaved mRNAs at GCU sequences. Notably, MqsR is a ribosome-independent mRNA interferase like MazF, which is distinctly different from ribosome-dependent mRNA interferases such as RelE (12,46), YoeB (18) and HigB (47).

It has been reported that MqsR is induced during biofilm formation (1) and by the addition of quorum-sensing autoinducer-2, AI-2 (2). The activation of MqsR, in turn, activates a two-component system, qseBC, which is known to play an important role in biofilm formation (2). QseC is a sensor histidine kinase and QseB is a transcription regulator, which binds to the 5′-UTR region of the qseBC operon and activates transcription of this operon (48,49). The MqsR-YgiT complex is able to bind two palindromic sequences present in the 5′-UTR of the MqsRYgiT operon and seems to repress transcription of the MqsRYgiT. We examined the possibility that the MqsR-YgiT complex may also regulate expression of the qseBC operon. However, the H-MqsR-YgiT complex was unable to bind the qseBC promoter region including the QseB binding site (data not shown). Both palindromic sequences (palindrome 1 and 2; FIG. 1A) were found to be unique on the E. coli chromosome, as there are no other E. coli genes other than the MqsRYgiT operon that have either of the two palindromic sequences. Also, purified QseB did not bind to the 5′-UTR region of the MqsRYgiT operon (data not shown). These results indicate that MqsR is not directly involved in the activation of the qseBC operon.

We analyzed all the 4,226 ORFs on the E. coli genome (NCBI RefSeq; accession No. NC 000091) for the existence of the GCU sequences and found that there are only 14 ORFs which do not contain a single GCU sequence (Table 3). Out of these 14 genes, six genes, pheL, tnaC, trpL, yciG, ygaQ and ralR have been shown to be induced during biofilm formation in E. coli (50). Of special interest is YgaQ (330 bp), which is induced 32 fold in biofilms and has also been shown to be involved in the swarming mobility of E. coli (51). Since these genes are resistant to MqsR mRNA inteferase activity, MqsR induction during biofilm formation may inactivate all E. coli mRNAs except for these 14 genes, which in turn may play an important role in biofilm formation. Almost all cells die during biofilm formation in Pseudomonas aeruginosa (52). MqsR induction during biofilm formation may cause the cells to enter a quasi-dormant state similar to that caused by MazF (8,53), and eventually lead to cell death.

The discovery of the MqsR-YgiT system as a new TA system in E. coli in the present paper increases the total number of the E. coli TA systems to as many as 16, which includes MazF-MazE (16,34), RelE-RelB (12,13), ChpBK-ChpBI (14), YafQ-DinJ (21), YoeB-YefM (18,19), HipA-HipB (22,23), HicA-HicB (25,26), YhaV-PrIF (27) and YafO-YafN (24).

TABLE 1
Primers used in this study

| Primer name | Sequence |
|---|---|
| MqsR Fw | 5′- TTTTTTTTTCATATGGAAAAACGCACACC |
| | ACATACAC -3′ |
| | SEQ ID NO: 5 |
| MqsR-Rv | 5′- TTTGAATTCTTACTTCTCCTTAAACGAGA |
| | CGATCAG -3′ |
| | SEQ ID NO: 6 |
| YgiT-Fw | 5′- TTTTTTTTTCATATGAAATGTCCGGTTTG |
| | C -3′ |
| | SEQ ID NO: 7 |
| YgiT-Rv | 5′- TTTGAATTCTTAACGGATTTCATTCAATA |
| | GTTCTGGATGC -3′ |
| | SEQ ID NO: 8 |
| RT-proF | 5′- TGCCTGACTCCAGCTTCCCTTA -3′ |
| | SEQ ID NO: 9 |
| RT-proR | 5′- TTAACGGATTTCATTCAATAGTTCTGGAT |
| | GC -3′ |
| | SEQ ID NO: 10 |
| RT-Fw | 5′- ACGCACACCACATACACGTT -3′ |
| | SEQ ID NO: 11 |
| RT-Rv | 5′- GCGAAAACGCATTTACACCT -3′ |
| | SEQ ID NO: 12 |
| YT-Rv | 5′- TTAACGGATTTCATTCAATAGTTCTGGAT |
| | GC -3′ |
| | SEQ ID NO: 13 |
| PX-RT | 5′- TGTATGTGGTGTGCGTTTTTCC -3′ |
| | SEQ ID NO: 14 |
| PX-F1 | 5′- TTGCCACCGTAACTGTTTTC -3′ |
| | SEQ ID NO: 15 |
| PX-F2 | 5′- TGTAACCCAGTGCATCATAAAC -3′ |
| | SEQ ID NO: 16 |
| PX-F3 | 5′- GCCAACACCGTCGCCGTTAGA -3′ |
| | SEQ ID NO: 17 |
| PX-F4 | 5′- TTTTTTACCGTTGCCAAGAGGT -3′ |
| | SEQ ID NO: 18 |
| PX-F5 | 5′- TGGCGAAGCCGCTGGTGTTTG -3′ |
| | SEQ ID NO: 19 |
| PX-F6 | 5′- GCCCACTTCAAAGTAGTTCA -3′ |
| | SEQ ID NO: 20 |
| PX-F7 | 5′- CATGTCGCCATTGCCACCGT -3′ |
| | SEQ ID NO: 21 |
| PX-F8 | 5′- CGAAAGAACCAACGTCAGCG -3′ |
| | SEQ ID NO: 22 |
| PX-F9 | 5′- GGTAGCAACGCCGCCAACAC -3′ |
| | SEQ ID NO: 23 |
| PX-F10 | 5′- GTTACGGGTTTCACCGTAG -3′ |
| | SEQ ID NO: 24 |
| PX-F11 | 5′- GCGCAACTAACAGAACGTCT -3′ |
| | SEQ ID NO: 25 |
| PX-F12 | 5′- TTCGGCATTTAACAAAG -3′ |
| | SEQ ID NO: 26 |
| PX-A | 5′- CAGTGTACCAGGTGTTATCTT -3′ |
| | SEQ ID NO: 27 |
| Px-Lp | 5′- AGCTGATCGATTTTAGCGTT -3′ |
| | SEQ ID NO: 28 |
| B3 | 5′- AGCACACCCACCCCGTTTAC -3′ |
| | SEQ ID NO: 29 |
| J | 5′- GGTTCAAGATACCTAGAGAC -3′ |
| | SEQ ID NO: 30 |
| D2 | 5′- TCTCTATTTATCTGACCGCG -3′ |
| | SEQ ID NO: 31 |
| E2 | 5′- TACAGGTTACTTTGTAAGCC -3′ |
| | SEQ ID NO: 32 |
| Palndrome | 5′- CCCCTAACTAACCTTTTAGGTGC |
| 1F | TTTTCCCC -3′ |
| | SEQ ID NO: 33 |
| Palindrome | 5′- GGGGAAAAGCACCTAAAAGGTTA |
| 1R | GTTAGGGG -3′ |
| | SEQ ID NO: 34 |
| Palindrome | 5′- CCCAATTAACCTTTTAGGTTATA |
| 2F | ACCC -3′ |
| | SEQ ID NO: 35 |
| Palindrome | 5′- GGGTTATAACCTAAAAGGTTAAT |
| 2R | TGGG -3′ |
| | SEQ ID NO: 36 |

TABLE 2
Cleavage sites of MqsR in vivo and in vitro

| | in vivo | | | in vitro |
|---|---|---|---|---|
| | ompA | ompF | lpp | MS2RNA |
| | ACA G▾CUAU | CCU G▾CU CU | AAA▾G▾CUAC | GAC▾G▾CUAG |
| | ATC G▾CGAU | CCU G▾CU CU | | CUC▾G▾CUGC |
| | CUG G▾CUGG | AAC▾G▾CU GC | | AGC▾G▾CUAC |
| | UUC G▾CUAC | GGC G▾CU GA | | UUC▾G▾CUAA |
| | | UAC G▾CU GA | | UUC▾G▾CUAC |
| | | GUU G▾CU AC | | AGA▾G CUUC |
| | | UUC G▾CU UC | | |
| | | UCA G▾CU AC | | |
| | | AAG G▾CU UU | | |
| | | GGU▾G▾CU UA | | |
| | | GCA▾G CU GA | | |
| | | GAA▾G CU CA | | |
| | | AAA▾G CU GA | | |
| | | UGG▾G CU AC | | |
| | | AUC▾G▾CU UA | | |
| | | AAC▾G CU AC | | |
| | | GCG▾G CU UC | | |
| weak | | AGC▾G CA AU | UGG G▾CGCG | |
| cleav- | | CUG▾G▾CA GU | | |
| age | | GUA G▾CA GG | | |
| | | AUG G▾CC UG | | |
| | | CGA G▾CG AG | | |
| | | UUG▾G CA AC | | |
| | | AAA G▾CG AA | | |

TABLE 3
The MqsR resistance genes in the E. coli genome

| | | | | Expected | Actual |
|---|---|---|---|---|---|
| | Length | | | Motif | Motif |
| Location | (bp) | Gene | Product | counts | counts |
| 339017 . . . 339313 | 297 | yahH | hypothetical protein | 4.64 | 0 |
| 736867 . . . 737121 | 255 | ybfQ | predicted transposase | 3.98 | 0 |
| 795195 . . . 795344 | 150 | ybhT | hypothetical protein | 2.34 | 0 |
| 1068503 . . . 1068676 | 174 | ymdF | hypothetical protein | 2.72 | 0 |
| Complement | 180 | yciG | hypothetical protein | 2.81 | 0 |
| (1317570 . . . 1317749) | | | | | |
| Complement | 45 | trpL | trp operon leader peptide | 0.70 | 0 |
| (1324752 . . . 1324796) | | | | | |
| Complement | 195 | ralR | restriction alleviation protein | 3.05 | 0 |
| (1415447 . . . 1415641) | | | | | |
| Complement | 222 | kilR | inhibitor of FtsZ, killing protein | 3.47 | 0 |
| (1419722 . . . 1419943) | | | | | |
| 2092133 . . . 2092183 | 51 | hisL | his operon leader peptide | 0.80 | 0 |
| 2736255 . . . 2736302 | 48 | pheL | pheA gene leader peptide | 0.75 | 0 |
| 2785053 . . . 2785385 | 333 | ygaQ | hypothetical protein | 5.20 | 0 |
| Complement | 75 | tnaC | tryptophanase leader peptide | 1.17 | 0 |
| (3751906 . . . 3751980) | | | | | |
| 4161624 . . . 4161824 | 201 | yheV | hypothetical protein | 3.14 | 0 |
| 4357586 . . . 4357759 | 174 | yjdO | hypothetical protein | 2.72 | 0 |

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

### REFERENCES

1. Balestrino, D., Haagensen, J. A., Rich, C., and Forestier, C. (2005) J Bacteriol 187, 2870-2880
2. Davies, D. G., Parsek, M. R., Pearson, J. P., Iglewski, B. H., Costerton, J. W., and Greenberg, E. P. (1998) Science 280, 295-298
3. Hammer, B. K., and Bassler, B. L. (2003) Mol Microbiol 50, 101-104
4. McNab, R., Ford, S. K., El-Sabaeny, A., Barbieri, B., Cook, G. S., and Lamont, R. J. (2003) J Bacteriol 185, 274-284
5. Ren, D., Bedzyk, L. A., Thomas, S. M., Ye, R. W., and Wood, T. K. (2004) Appl Microbiol Biotechnol 64, 515-524
6. Gonzalez Barrios, A. F., Zuo, R., Hashimoto, Y., Yang, L., Bentley, W. E., and Wood, T. K. (2006) J Bacteriol 188, 305-316
7. Pandey, D. P., and Gerdes, K. (2005) Nucleic Acids Res 33, 966-976
8. Yamaguchi, Y., and Inouye, M. (2009) Prog Mol Biol Transl Sci 85, 467-500
9. Buts, L., Lah, J., Dao-Thi, M. H., Wyns, L., and Loris, R. (2005) Trends Biochem Sci 30, 672-679
10. Engelberg-Kulka, H., Sat, B., Reches, M., Amitai, S., and Hazan, R. (2004) Trends Microbiol 12, 66-71
11. Gerdes, K., Christensen, S. K., and Lobner-Olesen, A. (2005) Nat Rev Microbiol 3, 371-382
12. Pedersen, K., Zavialov, A. V., Pavlov, M. Y., Elf, J., Gerdes, K., and Ehrenberg, M. (2003) Cell 112, 131-140
13. Takagi, H., Kakuta, Y., Okada, T., Yao, M., Tanaka, I., and Kimura, M. (2005) Nat Struct Mol Biol 12, 327-331
14. Zhang, Y., Zhu, L., Zhang, J., and Inouye, M. (2005) J Biol Chem 280, 26080-26088
15. Kamada, K., Hanaoka, F., and Burley, S. K. (2003) Mol Cell 11, 875-884
16. Zhang, J., Zhang, Y., and Inouye, M. (2003) J Biol Chem 278, 32300-32306
17. Zhang, Y., Zhang, J., Hara, H., Kato, I., and Inouye, M. (2005) J Biol Chem 280, 3143-3150
18. Zhang, Y., and Inouye, M. (2009) J Biol Chem 284, 6627-6638
19. Kamada, K., and Hanaoka, F. (2005) Mol Cell 19, 497-509
20. Motiejunaite, R., Armalyte, J., Markuckas, A., and Suziedeliene, E. (2007) FEMS Microbiol Lett 268, 112-119
21. Prysak, M. H., Mozdzierz, C. J., Cook, A. M., Zhu, L., Zhang, Y., Inouye, M., and Woychik, N. A. (2009) Mol Microbial 71, 1071-1087
22. Keren, I., Shah, D., Spoering, A., Kaldalu, N., and Lewis, K. (2004) J Bacteriol 186, 8172-8180
23. Korch, S. B., Henderson, T. A., and Hill, T. M. (2003) Mol Microbiol 50, 1199-1213
24. Brown, J. M., and Shaw, K. J. (2003) J Bacteriol 185, 6600-6608
25. Jorgensen, M. G., Pandey, D. P., Jaskolska, M., and Gerdes, K. (2009) J Bacteriol 191, 1191-1199
26. Makarova, K. S., Grishin, N. V., and Koonin, E. V. (2006) Bioinformatics 22, 2581-2584
27. Schmidt, 0., Schuenemann, V. J., Hand, N. J., Silhavy, T. J., Martin, J., Lupas, A. N., and Djuranovic, S. (2007) J Mol Biol 372, 894-905
28. Wang, X., Kim, Y., and Wood, T. K. (2009) ISMS J
29. Bahassi, E. M., O'Dea, M. H., Allali, N., Messens, J., Gellert, M., and Couturier, M. (1999) Biol Chem 274, 10936-10944
30. Kampranis, S. C., Howells, A. J., and Maxwell, A. (1999) J Mol Biol 293, 733-744
31. Christensen, S. K., and Gerdes, K. (2003) Mol Microbiol 48, 1389-1400
32. Hayes, C. S., and Sauer, R. T. (2003) Mol Cell 12, 903-911
33. Zhang, J., Zhang, Y., Zhu, L., Suzuki, M., and Inouye, M. (2004) J Biol Chem 279, 20678-20684
34. Zhang, Y., Zhang, J., Hoeflich, K. P., Ikura, M., Qing, G., and Inouye, M. (2003) Mol Cell 12, 913-923
35. Sarmientos, P., Sylvester, J. E., Contente, S., and Cashel, M. (1983) Cell 32, 1337-1346
36. Baker, K. E., and Mackie, G. A. (2003) Mol Microbiol 47, 75-88
37. Zhang, J., and Inouye, M. (2002) J Bacteriol 184, 5323-5329
38. Liu, Y. C., and Chou, Y. C. (1990) Biotechniques 9, 558, 560
39. Yoshida, T., Qin, L., Egger, L. A., and Inouye, M. (2006) J Biol Chem 281, 17114-17123
40. Guzman, L. M., Belin, D., Carson, M. J., and Beckwith, J. (1995) J Bacteriol 177, 4121-4130
41. Christensen, S. K., Pedersen, K., Hansen, F. G., and Gerdes, K. (2003) J Mol Biol 332, 809-819
42. Dao-Thi, M. H., Charlier, D., Loris, R., Maes, D., Messens, J., Wyns, L., and Backmann, J. (2002) J Biol Chem 277, 3733-3742
43. Madl, T., Van Melderen, L., Mine, N., Respondek, M., Oberer, M., Keller, W., Khatai, L., and Zangger, K. (2006) J Mol Biol 364, 170-185
44. Oberer, M., Zangger, K., Gruber, K., and Keller, W. (2007) Protein Sci 16, 1676-1688
45. Marianovsky, I., Aizenman, E., Engelberg-Kulka, H., and Glaser, G. (2001) J Biol Chem 276, 5975-5984
46. Li, G. Y., Zhang, Y., Inouye, M., and Ikura, M. (2008) J Mol Biol 380, 107-119
47. Hurley, J. M., and Woychik, N. A. (2009) J Biol Chem
48. Clarke, M. B., and Sperandio, V. (2005) Mol Microbiol 58, 441-455
49. Sperandio, V., Torres, A. G., and Kaper, J. B. (2002) Mol Microbiol 43, 809-821
50. Domka, J., Lee, J., Bansal, T., and Wood, T. K. (2007) Environ Microbiol 9, 332-346
51. Inoue, T., Shingaki, R., Hirose, S., Waki, K., Mori, H., and Fukui, K. (2007) J Bacteriol 189, 950-957
52. Webb, J. S., Thompson, L. S., James, S., Charlton, T., Tolker-Nielsen, T., Koch, B., Givskov, M., and Kjelleberg, S. (2003) J Bacteriol 185, 4585-4592
53. Inouye, M. (2006) J Cell Physiol 209, 670-676

1-54. (canceled)

55. A method of treating a patient with a disease requiring the inhibition of cell function comprising administering to the patient a mRNA interferase that cleaves mRNA at GCx, wherein x is A, C, G, or U, or a gene encoding said mRNA interferase.

56. The method of claim 55, wherein the mRNA is cleaved at GCU.

57. The method of claim 55, wherein the disease is cancer, bacterial infection or viral infection.

58. The method of claim 57, wherein the viral infection is caused by HIV.

59. The method of claim 57, wherein the viral infection is caused by a virus having a single-stranded RNA genome.

60. The method of claim 55, wherein the mRNA interferase is MqsR or a homolog thereof.

61. A method of cleaving mRNA comprising contacting an mRNA interferase that cleaves mRNA at GCx, wherein x is A, C, G, or U with mRNA.

62. The method of claim 61, wherein the mRNA is cleaved at GCU.

63. The method of claim 61, wherein the mRNA interferase is MqsR or a homolog thereof.

64. A method of producing a polypeptide having endoribonuclease activity comprising:
a. transforming a cell by introducing a polynucleotide encoding MqsR into the cell, and
b. culturing the transformed cell.

65. A method of producing a polypeptide having antitoxin activity comprising:
a. transforming a cell by introducing a polynucleotide encoding YgiT into the cell, and
b. culturing the transformed cell,
